(19) United States
(12) Patent Application Publication
(10) Pub. No.: US 2009/0240288 A1
(43) Pub. Date: Sep. 24, 2009

(54) IMPLANTABLE PLATE AND METHOD FOR ITS PRODUCTION

(75) Inventor: Richard Jean-Claude Guetty, Miribel (FR)

Correspondence Address: BRYAN CAVE POWELL GOLDSTEIN, ONE ATLANTIC CENTER FOURTEENTH FLOOR, 1201 WEST PEACHTREE STREET NW, ATLANTA, GA, 30309-3488, US

(73) Assignee: Compagnie De Recherche En Composants, Implants Et Materiels Pour L'Application Clinique, Vienne (FR)

(21) Appl. No.: 12/282,802

(22) PCT Filed: Mar. 14, 2007
(86) PCT No.: PCT/FR2007/000438

§ 371 Date: Feb. 26, 2009

(30) Foreign Application Priority Data
Mar. 14, 2006 (FR) 0602262

(51) Int. Cl. A61B 17/80

(52) U.S. Cl. 606/285

(57) ABSTRACT

An implantable reinforcement plate (1), comprising a first stable configuration having a first general shape curved substantially inwards on a first general curvature, wherein the plate can be deformed to change to a second stable configuration having a second general shape curved substantially inwards on a second general curvature, wherein the second general curvature is the inverse of the first general curvature.

### TECHNICAL FIELD

The present invention relates to the technical field of prosthetic implants, and more specifically to the field of surgical implants designed to limit the weakening or deterioration of a biological wall, such as the abdominal wall.

The present invention relates more specifically to an implantable reinforcement plate, in particular a plate to cure hernias, specifically inguinal hernias. The invention also relates to a production method for such an implantable reinforcement plate.

### PRIOR TECHNIQUE

The standard technique for curing abdominal hernias, in particular inguinal hernias, involves the surgical implantation of a prosthetic plate designed to repair and reinforce the inguinal region.

In particular, in the case of implantation of such a parietal reinforcement plate using a celioscopic approach or using an open posterior approach, successful treatment requires that the plate can conform to different internal anatomical contours that are not all on the same plane and which consist, in particular, of: Cooper's ligament, spermatic cord, iliac vessels, iliopsoas muscle, transverse muscle, fascia transversalis, conjoint tendon, rectus abdominis muscles, peritoneum and pubis.

To satisfy this requirement for anatomical conformity, parietal reinforcement plates have been proposed, formed by sewing two textile flaps along a serpentine flat line, so that the plate thus formed, once its flaps have been unfolded, has a conformation similar to that of the anatomical contours it is designed to cover.

Due to its “anatomical” construction, such a plate has an asymmetrical character, preventing it from being used indifferently for either the right or left side. It is therefore necessary, with a plate designed in this way, to have two distinct plates that are symmetrical to each another, with one being designed for the treatment of right inguinal hernias and having a geometry adapted for this purpose, and the other designed for the treatment of left inguinal hernias and having a geometry adapted for this purpose.

If these “anatomical” plates permit, to a certain extent, facilitation of the surgeon's work due to their geometry which is close to the internal anatomical contours they are designed to cover, they nonetheless have a certain number of disadvantages.

First of all, these known plates require a double production tool (or dual control of the production tool) to guarantee the manufacture of prostheses to cure right inguinal hernias and prostheses to cure left inguinal hernias. This complicates the organization of stock production and management, and contributes to increasing the price of the plate.

This also complicates institutional organization (of hospitals and clinics) using these plates, which must keep a permanent stock of right plates and left plates, in order to be able to treat all the various pathological conditions.

Aside from these logistical and economic problems, despite all the precautions taken, there may also exist a risk of confusion between a right plate and a left plate, with the all-negative medical consequences that such confusion is capable of engendering.

Finally, the two textile flaps of these known plates, in the absence of solicitation, are superimposed on one another, i.e. these known plates are not constructed to have an “anatomical” conformation. This lack of anatomical preforming is, of course, not likely to facilitate the surgeon's work, in particular when using a celioscopic approach.

### SUMMARY OF THE INVENTION

Therefore, the object of the objects assigned to the invention is to propose a new parietal reinforcement implantable plate not having the disadvantages cited above and permitting adaptation to several different anatomical configurations, in particular to symmetrical anatomical configurations.

Another object of the invention is to propose a new parietal reinforcement implantable plate whose conformation is particularly adapted to the anatomical contour it is designed to cover.

Another object of the invention is to propose a new parietal reinforcement implantable plate permitting particularly simple and easy management of its stock.

Another object of the invention is to propose a new parietal reinforcement implantable plate that eliminates all risks linked to the confusion between a right plate and a left plate.

Another object of the invention is to propose a new parietal reinforcement implantable plate with particularly simple and inexpensive construction.

Another object of the invention is to propose a new parietal reinforcement implantable plate that permits obtaining a plate capable of adapting to different anatomical configurations.

Another object of the invention is to propose a new production method for a parietal reinforcement implantable plate that is particularly simple, rapid and inexpensive to implement.

The objects assigned to the invention are achieved with the aid of a reinforcement implantable plate characterized in that it has a first stable configuration, in which it has a first general shape curved substantially inwards on a first general curvature, and in that it can be deformed in order to change into a second stable configuration, in which it has a second general shape curved substantially inwards on a second general curvature, which is the inverse of the first general curvature.

The objects assigned to the invention are also achieved with the aid of a production method for a reinforcement implantable plate characterized in that it includes a production phase of a plate having a first stable configuration, in which said plate has a general shape curved substantially inwards on a first general curvature, with said plate being made from a deformable material to be able to change into a second stable configuration, in which it has a general shape curved substantially inwards on a second general curvature, which is the inverse of the first general curvature.

### SUMMARY SPECIFICATIONS OF THE INVENTION

Other objects and benefits of the invention will become clearer upon reading the attached description, in addition to the help of the attached drawings, purely by way of illustration and for informational purposes, which include:

FIG. 1 is a perspective view, according to a lateral viewpoint, of a reinforcement implantable plate conforming to a first embodiment of the invention, in its first configuration, with said plate comprising in this case a plate for the cure of a right inguinal hernia.

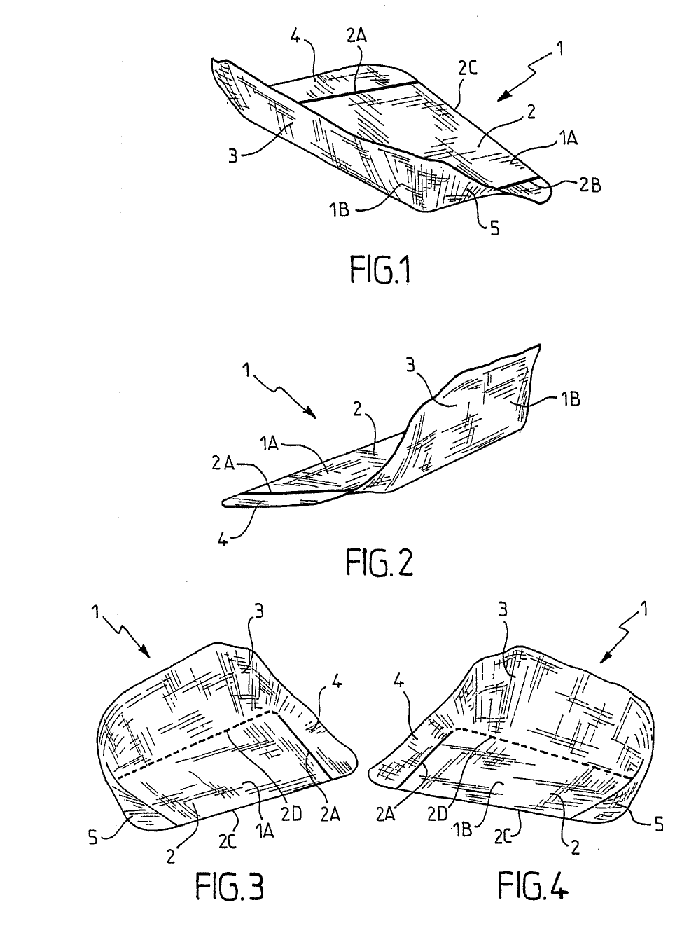

FIG. 2 is a perspective view, according to a lateral point of view substantially opposed to that of FIG. 1, the plate in FIG. 1 in its first configuration.

FIG. 3 illustrates, according to a top perspective view, the implantable plate of FIGS. 1 and 2, in its first configuration.

FIG. 4 illustrates, according to a top perspective view, the implantable plate of FIGS. 1 to 3, in its second configuration, with said plate comprising in this case a plate for curing a left inguinal hernia.

FIG. 5 illustrates, according to a top view and adhering to proportions, a flat textile piece designed for fabrication of the plate illustrated in FIGS. 1 to 4.

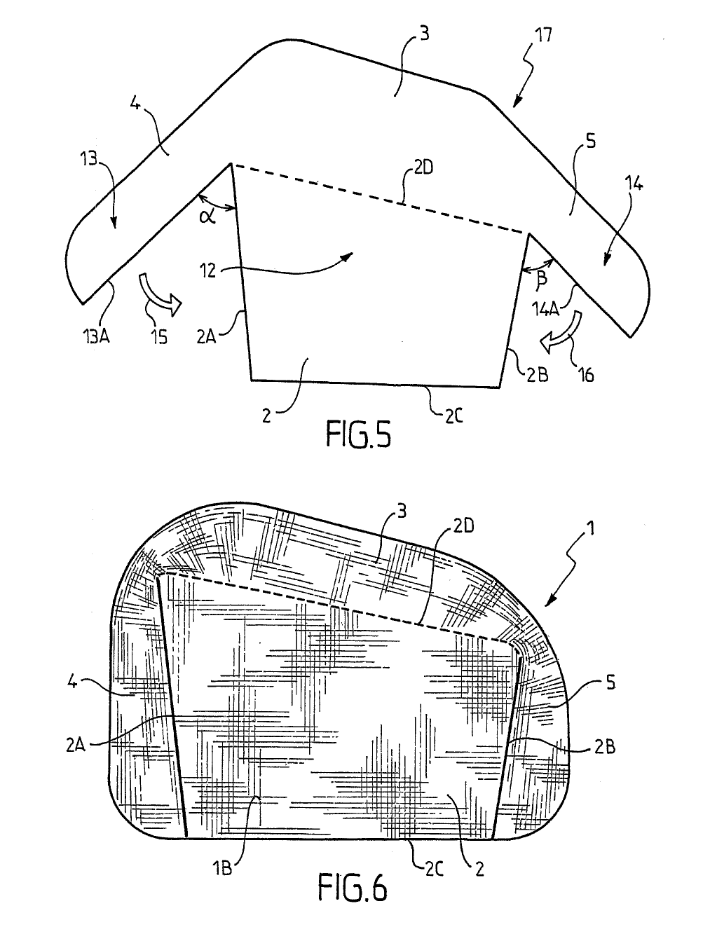

FIG. 6 illustrates, according to a top view, the plate from FIGS. 1 to 4 in its second configuration, obtained from the textile piece illustrated in FIG. 5.

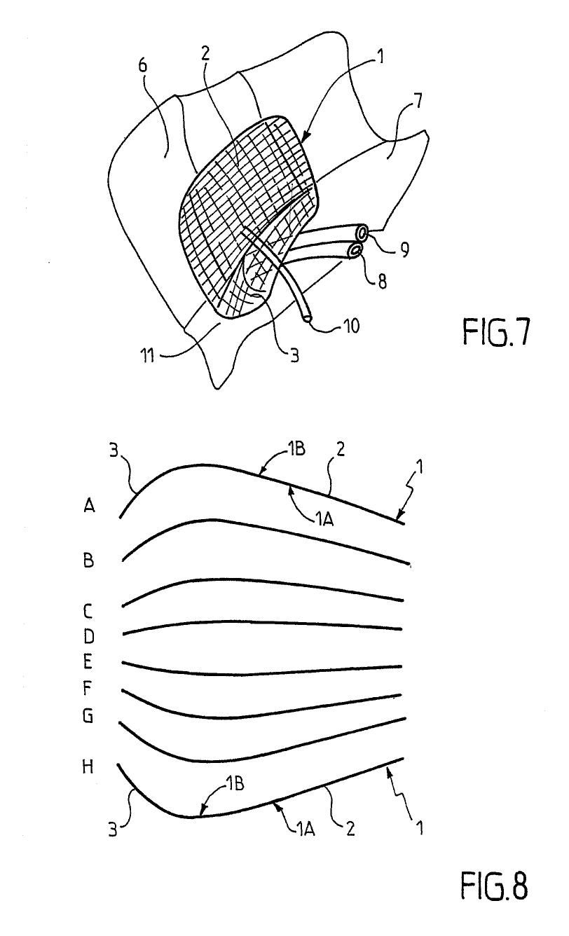

FIG. 7 illustrates, according to a schematic perspective view, the anatomical positioning, for treatment of a right inguinal hernia, of the plate illustrated in FIGS. 1 to 4 and 6 in its first configuration.

FIG. 8 illustrates, according to a sectional schematic view, the change of the plate, broken down into six intermediate states B-G, illustrated in FIGS. 1 to 4, 6 and 7, from its first stable configuration (state A) to its second stable configuration (state H).

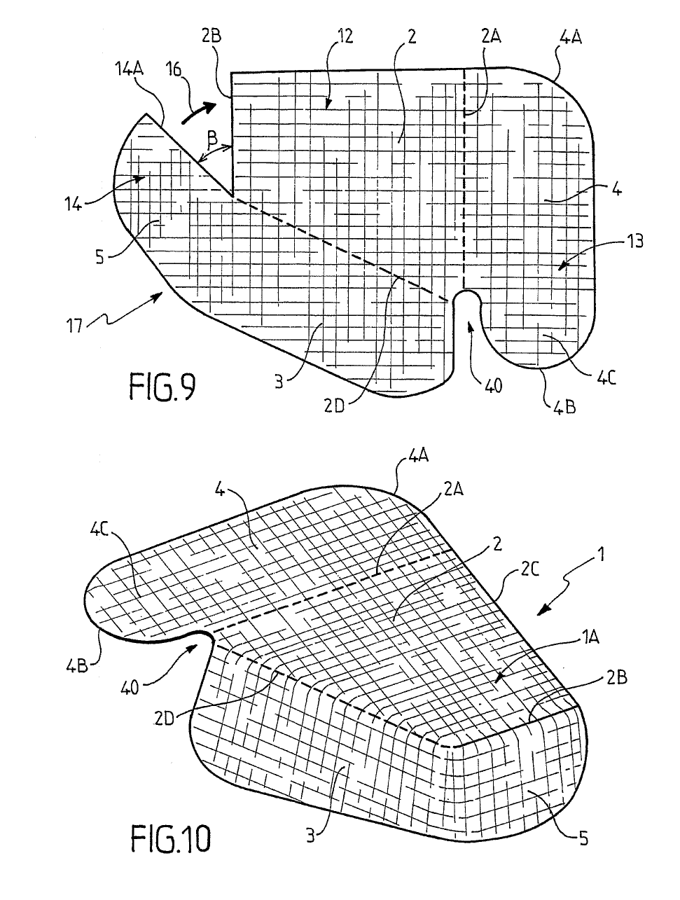

FIG. 9 illustrates, according to a top view and adhering to proportions, a flat textile piece designed for the production of a plate conforming to a second embodiment of the invention.

FIG. 10 illustrates, according to a top view, the plate obtained from the textile piece illustrated in FIG. 9, with said plate in its second configuration.

### BEST METHOD OF PRODUCTION FOR THE INVENTION

The invention relates to a reinforcement plate 1, designed to be surgically placed in order to reinforce, or repair, a weakened or damaged biological organ. Advantageously, plate 1 is a parietal reinforcement implantable plate, i.e. it has been specifically designed to reinforce the biological wall, for example muscular, and preferably the abdominal wall. Plate 1 conforming to the invention may therefore be advantageously designed to treat hernias or eventrations.

In the two preferable variants illustrated in the figures, plate 1 consists of a plate to cure an inguinal hernia. Even more preferably, plate 1 consists of a plate to cure an inguinal hernia treated by a celioscopic approach (preferably pre-peritoneal) or by an open posterior approach.

In what follows, for the sake of concision and simplicity, only a plate 1 to cure inguinal hernia treated by a celioscopic approach will be described. The invention is however, in no way limited to such a plate, and relates also to other plates (eventration in particular), independent of their method of placement (celioscopic or open approach), without leaving the framework of the invention.

The term “plate” should be understood in its currently accepted meaning in the technical field under consideration, i.e. designating a prefabricated solid prosthetic implant, as opposed to a gel, cream, or lotion, for example.

In accordance with the invention, the implantable reinforcement plate 1 has a first stable configuration (illustrated in FIGS. 1 to 3 and 7), in which it has a first shape curved substantially inwards on a first general curvature.

Stable configuration refers to a configuration maintaining itself, i.e. one that has its own mechanical strength. In other terms, plate 1 is preformed to provide this first stable configuration, i.e. through its construction it presents this first stable inwardly curved configuration, without any external solicitation or constraint being necessary to maintain its inward curvature.

In its first stable configuration, plate 1 presents, as indicated, a first general shape curved substantially inward, i.e. substantially hollow, concave, on a curved profile designated here by the expression “general curvature”. In other words, plate 1 has, in its first stable configuration, an inward curvature inscribed within a curved surface, corresponding to the meeting of all the curvature lines of plate 1 and which is globally designated here by the term “general curvature.”

In conformity with the invention, plate 1 is deformable to be able to change into a second stable configuration (illustrated in FIGS. 4, 6 and 10), in which it has a second general shape curved substantially inwards on a second general curvature, which is the inverse of the first general curvature. In other terms, plate 1 is capable of changing from its first stable configuration to its second stable configuration by inversion of its inward curvature, i.e. inversion of the first general curvature so as to obtain the second general curvature. Plate 1 can also change from its first configuration to its second configuration by folding back on itself.

For this reason, plate 1 is made in a material sufficiently flexible to allow for inversion of the curvature of plate 1, so that the plate, following inversion of its curvature, is distinct, in its second stable configuration, from its first configuration.

The deformation method of plate 1 from its first stable configuration to its second stable configuration is illustrated schematically in FIG. 8, which schematically illustrates plate 1 in six intermediate states of deformation referenced B to G through which plate 1 changes successively from its first configuration (referenced A) to its second configuration (referenced H). A progressive decrease in the inward curvature of prosthesis 1 from its first stable configuration A to state of deformation D is thus illustrated in FIG. 8. Between state of deformation D and state of deformation E the general curvature of plate 1 is inversed, so that plate 1 now has an inward curvature opposed to that it manifested in its first stable configuration A. The inversed inward curvature increases from state F to state G, until plate 1 demonstrates its second stable configuration H.

Of course, the illustration in FIG. 8 is purely schematic and it is perfectly conceivable, without leaving the framework of the invention, that plate 1 will change, from its first stable configuration A to its second stable configuration H, through intermediate states of deformation, whose geometry differs significantly from the one illustrated, solely by way of example, in FIG. 8.

Preferentially, the deformable material from which plate 1 is made permits manual deformation of plate 1 from its first stable configuration to its second stable configuration. In this way, the surgeon can immediately, preferably without any particular tool, very easily modify plate 1 so that it adopts its second stable configuration, if the surgical situation requires it.

The general principle of the invention is based on implantation of a single plate that can adopt, in a stable fashion, at least two different geometric configurations, with each of these geometric configurations thus capable of being suited to a specific surgical situation.

In the examples illustrated in the figures, and as will be described more fully in what follows, the first stable configuration corresponds to a cure for a right inguinal hernia, whereas the second stable configuration corresponds to a cure for a left inguinal hernia. Therefore, when it is in its first configuration, plate 1 is conformed to substantially follow the anatomical contour corresponding to a right inguinal hernia, whereas in its second configuration, it is conformed to substantially follow the anatomical contour corresponding to a left inguinal hernia.

Beneficially, plate 1 is designed to go from its first stable configuration to its second stable configuration by being reversible, that is that its user may at will conform plate 1 in the first stable configuration or in the second stable configuration. This enables a substantial flexibility and safety in use, as the practician (or those assisting) therefore maintain permanent control of the configuration (left or right, in the case illustrated in the figures) of plate 1.

Advantageously, as illustrated in the figures, plate 1 conforming to the invention has a first side 1A and a second opposed side 1B, with said first and second sides 1A, 1B being separated by the thickness of plate 1. Advantageously, as illustrated in the figures, in the first stable configuration, the first side 1A is substantially concave, whereas the second side 1B is substantially convex, whereas in the second configuration, following inversion of the curvature of plate 1, the first side 1A is substantially convex, whereas the second side 1B is substantially concave.

Advantageously, plate 1 conforming to the present invention is asymmetrical in at least one of its stable configurations. In other words, the first general shape is preferably substantially asymmetrical and/or the second general shape is substantially asymmetrical. In the preferred embodiments of the invention illustrated in the figures, the first and second general shapes are both substantially asymmetrical, i.e. plate 1 remains asymmetrical independent of the stable geometric configuration that it may have.

In the examples illustrated in the figures, plate 1 curing a hernia thus has a principal flap 2, whose form is substantially flat and quadrangular, extending between a first lateral edge 2A (exemplified by a fictitious dotted line in FIGS. 9 and 10) and a second lateral edge 2B and additionally an upper edge 2C and a lower edge 2D (the latter exemplified by a fictitious dotted line in FIGS. 3 to 6, 9 and 10). The lateral edges 2A, 2B, upper 2C and lower 2D are preferably substantially rectilinear, with the first lateral edge 2A being preferably of a substantially greater length than that of the second lateral edge 2B, whereas the superior edge 2C is preferably substantially shorter than the lower edge 2D.

Advantageously, a second flap 3 extends from lower edge 2D substantially obliquely relative to principal flap 2. Secondary flap 3 thus forms a spatula for principal flap 2. This angular gap between principal flap 2 and secondary flap 3, resulting from construction, obviously helps to confer its inward curving character to plate 1, with the maximal inward curving zone corresponding preferably to the junction zone between lower edge 2D and secondary flap 3.

In the first variant of FIGS. 1 to 8, secondary flap 3 is laterally extended by two lateral flaps 4, 5 which extend respectively from the first and second lateral edge 2A, 2B of principal flap 2. Each lateral flap 4, 5 thus links secondary flap 3 respectively to the first and second lateral edge 2A, 2B.

In the second variant of FIGS. 9 and 10, the principal flap 2 is extended, from its first lateral edge 2A, by a first lateral flap 4 extending preferably substantially in the same plane as the plane of extension of the principal flap 2. The first lateral flap extends:
laterally between a first lateral side corresponding to edge 2A and a second opposing lateral side,
and longitudinally between a back side 4A located in the extension of edge 2C and an opposite frontal rounded side 4B, preferably substantially semi-circular in shape.

According to this second variant, secondary flap 3 extends laterally via a single first lateral flap 5 which itself extends from the second lateral edge 2B of the principal flap 2. The first lateral flap 5 of this second variant thus links the secondary flap 3 to the second lateral edge 2B. As illustrated in FIGS. 9 and 10, a space 40 is provided between the secondary flap 3 and the first lateral flap 4, with said space 40 being designed to permit the passage of elements from the spermatic cord. The extreme zone 4C of flap 4 located toward frontal side 4B is designed to be pierced through with a clip, so as to be clipped to Cooper ligament 11.

Advantageously, secondary flap 3 has a substantially asymmetrical form, adapted to optimally cover the elements of the spermatic cord. For this purpose, secondary flap 3 has preferably a significantly larger area to the right of first lateral edge 2A than to the right of second lateral edge 2B. In other words, the width of the secondary flap 3 is preferentially greater toward the first lateral edge 2A than toward the second lateral edge 2B.

As illustrated in FIG. 7, the specific geometry just described permits excellent adaptation to the anatomical contour of the inguinal area. In the case of a cure for a right inguinal hernia, as represented in FIG. 7, principal flap 2 acts as a support for the anterior abdominal muscle wall 6, while secondary flap 3 substantially follows, and preferably conforms to the contours formed by the iliopsoas muscle 7, the external iliac vessels 8, the testicular vessels 9, the vas deferens and Cooper's ligament 11.

In order to follow the anatomical contour previously described as closely as possible, it is preferable that plate 1 be constructed so that it is permanently substantially flexible and flaccid. For this reason, plate 1 preferably includes a textile mat and even more preferably is substantially fully comprised by such a textile mat, which is advantageously one-piece and sewn on it to give it an inwardly curved character.

Preferentially, the textile mat is a mat made of gridded polyester thread. It is however conceivable, without leaving the framework of the invention, that the textile mat can be made from threads or fibers of any other type, for example from polypropylene, monofilament or multifilament threads. The invention is also not limited to a specific production method for the textile mat, which can be woven, unwoven, braided or knitted, for example. Obviously, plate 1 is not necessarily comprised of a textile mat, but can include or be comprised of a synthetic film, for example.

Advantageously, the first general form presented by plate 1 in its first stable configuration is substantially symmetrical to the second general form presented by plate 1 in its second stable configuration, as demonstrated in particular by FIGS. 3, 4 and 8.

In other terms, in this particularly beneficial case, first side 1A in the first stable configuration is symmetrical to second side 1B in the second stable configuration, whereas second side 1B in the first stable configuration is symmetric to first side 1A in the second stable configuration. Because of this characteristic, which proves particularly interesting in the case of a plate designed to treat a pathological condition capable of appearing in two distinct sites in the human body that are symmetrical to each other, a single plate may be used, with its first stable configuration being geometrically adapted to the first site, whereas its second stable configuration is geometrically adapted to the second site, which is symmetrical to the first site.

Thus, plate 1, a cure for inguinal hernia, may be used, in its first stable configuration illustrated in FIG. 1, to treat a right inguinal hernia, as illustrated in FIG. 7.

The same plate 1 may also be used to treat a left inguinal hernia (not represented), since it is sufficient to have plate 1 change to its second stable configuration, by inversion of the concavity. A single and unique preformed plate 1 therefore enables a right or left inguinal hernia to be treated, whereas according to the prior state of the art, two distinct plates were required.

Plate 1 conforming to the invention may obviously be obtained using any production method known in the field. For example, plate 1 may be etched or thermally preformed (thermoforming), in order to give it its stable inwardly curving nature.

However, the stable inward curving of plate 1 is preferably obtained by a simple assembly operation.

Thus, the invention preferably relates to a plate 1 containing a central section 12 having at least a lateral edge 2A, 2B and from which respectively at least an arm 13, 14 extend, with said arm 13, 14 being flattened out against the corresponding lateral edge 2A, 2B, and being attached to the latter, preferably by sewing.

For example, as is evident more specifically in FIG. 5, plate 1 of the first variant includes advantageously a central section 12 forming principal flaps 2 and secondary 3. This central section 12 extends laterally between the first and second lateral edge 2A, 2B, from which respectively extend a first and second arm 13, 14 respectively corresponding to the lateral flaps 4, 5. Arms 13, 14 are flattened out respectively against the first and second lateral edge 2A, 2B (arrows 15, 16 in FIG. 5) and are attached to it by any appropriate method, preferably sewing. This has the effect of introducing a curvature in the central section 12, leading to the formation and differentiation of principal flaps 2 and secondary 3.

Advantageously, the central section 12, first arm 13 and second arm 14 form together the same piece 17 as a single piece, i.e. the first and second arm 13, 14 form part of central section 12.

Preferably, as illustrated in FIG. 5, the unique piece formed by central section 12, the first arm 13 and second arm 14 is a one-piece unitary textile piece with a three-lobed substantially flat form (clover-leaf) before being fixed to arms 13, 14 at corresponding lateral edges 2A, 2B. In this three-lobed form, the central lobe corresponds to central section 12, whose lateral edges 2A, 2B are substantially rectilinear, while the external lobes surrounding the central lobe correspond respectively to the first and second arm 13, 14. Each arm 13, 14 has a respective rectilinear binding edge 13A, 14A, substantially equal in length to that of the corresponding lateral edge 2A, 2B, to which it is designed to be sewn.

Each binding edge 13 A, 14A is respectively adjacent to the first and second lateral edge 2A, 2B and forms with said corresponding lateral edge 2A, 2B a corresponding non-negative angle α, β, preferably less than 90°. Given that the central section 12, and the first and second arms 13, 14 are all located, before being assembled, on the same plane (as illustrated by FIG. 5), the operation of attaching the attachable edges 13A, 14A to the corresponding lateral edges 2A, 2B will lead to inward curving of the textile piece and formation of the principal flap 2, secondary flap 3 and lateral flaps 4, 5.

In this embodiment of FIGS. 1 to 8, plate 1 is particularly stable, since the only geometric irregularities it has are the two seams made along each lateral edge 2A, 2B, with these seams extending, when plate 1 is in position (cf. FIG. 7), substantially facing the muscle wall 6, and not against particularly fragile and sensitive organs such as the external iliac vessels 8, testicular vessels 9 and the vas deferens 10, as was the case with the prior art.

In the second embodiment of FIGS. 9 and 10, the principle of construction is similar to that implemented for the first variant of FIGS. 1 to 8, with the difference being that the final form of the prosthesis is obtained by flattening out and attaching a single binding edge 14A to edge 2B, with said binding edge 14A being adjacent to edge 2B and forming with it an a non-negative angle β preferably less than 90°. After assembly, the secondary flap 3 and second lateral flap 5 are bent forward, whereas the first lateral flap 4 remains substantially in the plane of principal flap 2.

Finally, the invention relates to a production method as such for a reinforcement implantable plate, and in particular a reinforcement implantable plate corresponding to what has been described above. Advantageously, the method conforming to the invention comprises a production method for a plate to cure an inguinal hernia.

The method conforming to the invention thus includes a production phase of plate 1 having a first stable configuration, in which plate 1 has a general shape curved substantially inwards on a first general curvature. In this production phase, the plate is made from deformable material to permit it to change into a second stable configuration, in which said plate 1 has a general shape curving substantially inward on a second general curvature, which is the inverse of the first general curvature.

Advantageously, the production phase of plate 1 includes a cutting stage, in a membrane made from said deformable material, of a piece 17 having at least one notch with two adjacent edges (2A, 13A, 2B, 14A) forming a non-negative angle between them.

Advantageously, as illustrated in FIGS. 5 and 9, the membrane (and therefore piece 17) is substantially flat.

Thus, in the examples illustrated in the figures, the production phase includes a cutting stage, in a membrane made from said deformable material, of piece 17 (represented in FIGS. 5 and 9) containing a central section 12 extending laterally between a first and a second lateral edge 2A, 2B, from which respectively extend a first and second arm 13, 14, preferably in a substantially oblique manner.

Advantageously, the production phase includes in addition an inwardly curving stage during which said adjacent edges are flattened out and attached to each other, so as to give piece 17 an inwardly curving character.

Thus, in the examples illustrated in the figures, the production phase includes an inwardly curving stage during which arms 13, 14 are flattened out respectively against the first and second lateral edge 2A, 2B and are attached to the latter.

Preferably, the membrane is formed from a textile mat, whose adjacent edges (2A, 13A, 2B, 14A) are attached to each other by being sewn during the inwardly curving stage.

In the case of the examples illustrated in the figures, the first and second arm 13, 14 are respectively attached to the first and second edge 2A, 2B by being sewn, during the inwardly curving stage.

The invention thus permits to easily obtain an inwardly curving prosthetic plate, since the essential part of the plate manufacture consists of obtaining a single textile piece 17 through simple cutting (in three-lobar form) in the example of FIGS. 1 to 8), and then of flattening out arms 13, 14 of this single textile piece on the corresponding edges 2A, 2B of the central section 12, automatically causing the inward curving of the flat textile piece 17, which thus becomes the “reversible” plate 1 conforming to the invention.

### POSSIBILITY FOR INDUSTRIAL APPLICATION

The industrial application of the invention is found in the design, manufacture, and use of prosthetic plates, in particular for the treatment of hernias.

1. An implantable reinforcement plate, comprising: a first stable configuration having a first general shape curved substantially inwards on a first general curvature, wherein the plate can be deformed to change into a second stable configuration having a second general shape curved substantially inwards on a second general curvature, wherein the second general curvature is the inverse of the first general curvature.

2. The plate of claim 1, wherein either the first general shape is substantially asymmetrical or the second general shape is substantially asymmetrical.

3. The plate of claim 1, wherein the plate is reversibly changeable from the first configuration to the second configuration.

4. The plate of claim 1, wherein the first general shape is substantially symmetrical to the second general shape.

5. The plate of claim 1, wherein the plate presents a first side and a second side opposing the first side, wherein, in the first configuration, the first side is substantially concave while the second side is substantially convex, and wherein, in the second configuration, the first side is substantially convex while the second side is substantially concave.

6. The plate of claim 1, further comprising a central section having at least one lateral edge and from which respectively at least one arm extends, said arm is flattened out against the corresponding lateral edge and is attached to the corresponding lateral edge, preferably by sewing.

7. The plate of claim 1, further comprising a textile mat.

8. The plate of claim 7, wherein the textile mat is sewn on the plate in a single piece to give the plate an inwardly curving nature.

9. The plate of claim 1, further comprising a plate to cure an inguinal hernia.

10. The plate of claim 9, wherein the plate, in its first configuration, is conformed to substantially follow the anatomical contour corresponding to a right inguinal hernia, and wherein the plate, in its second configuration, is conformed to substantially follow the anatomical contour corresponding to a left inguinal hernia.

11. A method for producing an implantable reinforcement plate, comprising: producing a plate having a first stable configuration, in which said plate has a general shape curved substantially inwards on a first general curvature, with said plate being capable of being deformed in order to change into a second stable configuration, in which it presents a general shape curved substantially inwards on a second general curvature, which is the inverse of the first general curvature.

12. The method of claim 11, further comprising:
a) cutting, in a membrane made from said material capable of being deformed, in a piece having at least one notch with two adjacent edges forming a non-negative angle between them, and
b) inwardly curving said adjacent edges so that said adjacent edges are flattened out and attached to each other, so as to give the piece an inwardly curving shape.

13. The method of claim 12, wherein said membrane is substantially flat.

14. The method of claim 12, wherein said membrane is made of a textile mat whose adjacent edges are attached to one another by sewing during the inwardly curving stage.

15. The method of claim 11, wherein said method comprises a production method for a plate to cure an inguinal hernia.
